(19) United States
(12) Patent Application Publication
(10) Pub. No.: US 2012/0040312 A1
(43) Pub. Date: Feb. 16, 2012

(54) Dental Ultrasonography

(75) Inventor: Mark K. Hinders, Williamsburg, VA (US)

(73) Assignee: College of William and Mary, Williamsburg, VA (US)

(21) Appl. No.: 13/198,823

(22) Filed: Aug. 5, 2011

Related U.S. Application Data
(60) Provisional application No. 61/372,571, filed on Aug. 11, 2010.

(51) Int. Cl. A61B 8/13

(52) U.S. Cl. 433/215

(57) ABSTRACT

Methods and devices are described that enable real-time, hand-held diagnostic ultrasonography for use in dental practice. The methods and devices utilize circumferential surface wave modes that are highly sensitive to small surface and near surface flaws. In addition, these surface waves can propagate between the teeth, following the curvature of the tooth.

### CROSS-REFERENCE TO RELATED APPLICATIONS

This application claims the benefit of and priority to U.S. Provisional Patent Application Ser. No. 61/372,571, filed Aug. 11, 2010, the entire disclosure of which is incorporated by reference herein.

### STATEMENT REGARDING FEDERALLY SPONSORED RESEARCH AND DEVELOPMENT

Not applicable.

### FIELD OF INVENTION

The field of the invention relates to diagnostic ultrasonography in dentistry.

### BACKGROUND OF THE INVENTION

Megahertz-frequency ultrasound is a common medical diagnostic tool, but it has never been used routinely in the practice of dentistry. The terms “ultrasound” and “ultrasonic”, when used in the field of dentistry, typically refer to kilohertz-frequency vibrating tips used for scaling teeth and do not refer to diagnostic imaging as in medical diagnostics and industrial inspection. Although ultrasonography of dental tissues was first attempted in the early 1960s, it has not yet become a diagnostic tool in the clinical practice of oral health management. This long history of dental ultrasonography, in the absence of significant product development, indicates the difficulty of the challenges involved. Key challenges relating to teeth include tissue anisotropy and complex geometries of tooth surfaces. Additionally, imaging hard tissue such as teeth is significantly more complex than imaging soft tissue. There is a variety of different wave modes that can propagate in hard tissues whereas only the relatively simple longitudinal modes propagate in soft tissue. Over the last forty years, a variety of techniques have been investigated in vitro for applying ultrasonic evaluation to the tooth and surrounding tissue, including scanning acoustic microscopy and pulse-echo tooth-layer characterization. However, most current techniques utilizing ultrasound require the teeth to be extracted, ground or sectioned.

Diagnostic ultrasonography is common for other parts of the body. In B-mode medical imaging, a real-time display is presented for the portion of the anatomy within the field of view of the transducer. That image is interpreted by the health care professional as the probe is moved about to cover the anatomy of interest, e.g., the heart or the womb. Unlike these other parts of the anatomy, teeth cannot be scanned in this way because the interproximal features are inaccessible due to adjoining teeth. Also, it would be impractical in dental practice to systematically scan all teeth because of the time it would take to cover each one with the necessary resolution.

Ultrasonography is often a preferred diagnostic tool because it is safe, portable, easy to use, inexpensive, and capable of providing real-time data. It is also readily adaptable to specialized diagnostic applications, since interchangeable probes can be used with the same basic instrumentation. Ultrasonography is non-ionizing and entirely non-invasive, but has not yet come into widespread use in the dental office, primarily because of difficulty in coupling the ultrasound energy into and back out of the complex dental anatomy in a controlled way.

Ultrasound may be able to provide much earlier diagnosis of caries, even at the precarious stage when the lesion can be remineralized rather than drilled and filled. Ultrasound also has the advantage over manual periodontal probing via a graduated metal pick for gum disease. Ultrasound is utterly painless and could provide much better resolution than the +/−1 mm generously ascribed to the current diagnostic gold standard. Cracks in teeth which are almost never detectable via x-rays could be easily detected with ultrasound.

Accordingly, there is a need in the art for methods and devices for improving the imaging of oral diseases and reducing the use of potentially harmful x-rays in routine dental practice by adapting well-established medical ultrasonography into a high resolution imaging modality with enhanced specificity and sensitivity for early detection of oral diseases and for monitoring treatment outcome.

### BRIEF SUMMARY OF THE INVENTION

Methods and devices are described herein that enable real-time, hand-held diagnostic ultrasonography for detection of: demineralization of the enamel and dentin, demineralization or caries under and around existing restorations, caries on occlusal and interproximal surfaces, cracks of enamel and dentin, calculus, periapical lesions, etc. One novelty of this approach is the use of surface wave modes which are highly sensitive to small surface and near surface flaws. In addition, these surface waves can propagate between the teeth, following the curvature of the tooth. The waves can examine interproximal sites while the transducer remains in a more accessible location on the surface of the tooth.

One object of the invention relates to a device for ultrasonic imaging of teeth. The device contains a delay line transducer. The delay line comprises a flat middle portion in contact with a tooth and two angled sides connected to said flat middle portion, wherein the two angled sides are symmetric and form the same angle with said flat middle portion. Additionally, a compliant gel boot is in direct contact with said two angled sides and comprises an aqueous material. The ultrasound transducer emits an ultrasound wave onto the flat middle portion and the angled sides such that a wave is created which circumferentially travels the surface of said tooth. The taper of the angled sides is selected to generate such circumferential waves.

Another object of the present invention relates to a method for ultrasonic imaging of teeth. The method comprises passing a megahertz frequency through an interface of two materials with different indices of refraction. This generates counter-propagating circumferential surface waves which follow the surface of the tooth in vivo. Surface and subsurface defects in said tooth are identified by analyzing the echo from said surface waves.

The methods and devices described herein solve the technical challenge of achieving efficient coupling of ultrasound energy into and then back out of the tooth. In particular, these methods and devices generate surface wave modes that are most sensitive to surface flaws and sub-surface flaws and can be exploited to detect such flaws at interproximal sites when a ultrasonic probe tip contacts the tooth surface at easily accessible locations.

### BRIEF DESCRIPTION OF THE DRAWINGS

The summary above, and the following detailed description, will be better understood in view of the drawings which depict details of preferred embodiments.

FIG. 1 is a schematic drawing of a device for ultrasonic imaging of teeth.

FIG. 2 is a schematic drawing of a device for ultrasonic imaging of teeth, showing in greater schematic detail the paths of the ultrasonic waves.

FIG. 3A is a graph comparing ultrasonic waveforms of different regions of a tooth, one of which has a cavity and the other does not. FIG. 3B shows a slice of a CT-scan of a flawed tooth (with the flaw marked by an arrow). FIG. 3C shows Dynamic Wavelet Fingerprint (“DWFP”) comparison of the flawed and unflawed locations on the tooth. FIG. 3D is a graph showing the ridge counts as a function of depth for flawed and unflawed tooth sites, which provides a metric to discern the presence of flaws in the ultrasonography echoes.

FIG. 4 shows DWFP fingerprints corresponding to a filtered waveform from a phantom tooth with flaw (top), a filtered waveform from an unflawed phantom tooth (middle), and, at bottom, a graph showing ridge counts as a function of time for the flawed and unflawed teeth.

FIG. 5 shows two graphs comparing the average waveforms over nine sites for both flawed and unflawed examples. The lower (bottom) graph depicts a zoomed-in region from the top graph.

FIG. 6 is a graph showing the waveform comparison from flawed and unflawed sites of the same human tooth.

FIG. 7A is a graph showing the Rayleigh wave reflection in an aluminum plate, and FIG. 7B shows the corresponding DFWP.

FIG. 8 is a series of graphs showing five different measurement locations, with the corresponding Rayleigh wave reflections, for a flat sample with a slot flaw.

FIG. 9 shows three wavelet fingerprints with corresponding waveforms showing Rayleigh wave reflections from cracks in a curved ceramic sample at three different distances from the transducer.

### DETAILED DESCRIPTION OF THE INVENTION

The present invention is directed to diagnostic ultrasonography in the field of dentistry. Unless otherwise specified, the terms ultrasonography, ultrasound, and ultrasonographic refer to MHz-frequency ultrasound techniques.

The key technical challenge is to ensure efficient coupling of ultrasound energy into and then back out of the tooth. Methods and devices for doing so are described below.

As used herein, the term “compliant gel boot” refers to a flexible covering for the delay line tip which contains an ultrasonic coupling medium of the type known in the art such as those used for intraoral or intracavity ultrasound imaging. These are gels used to reduce the impedance mismatch between the transducer and the object being studied. For example, a suitable ultrasonic coupling gel is Clear Image, available from Sonotech Inc., of Bellingham, Wash. Ultrasonic coupling gels can be non-aqueous or, alternatively, can be water-based gels, including, but not limited to, water-based gels containing polyvinyl alcohol, polyvinyl pyrrolidone, polyethylene oxide, propylene glycol, and/or glycerin. Suitable probe covers are known in the art, and can be latex or non-latex (e.g., polyethylene). For example, suitable probe covers can be purchased from Microtek Medical of Columbus, MS. Alternatively, the compliant gel boot may instead utilize a conforming gel pad, which utilizes a solid ultrasound conductivity gel. Another suitable embodiment that does not require a probe cover is the use of a couplant sheet, which is a gel in a sheet form, and can be purchased, for example, from Civco Medical Solutions of Kalona, Iowa.

When an ultrasonic wave passes through an interface between two materials at an oblique angle, and the materials have different indices of refraction, both reflected and refracted waves are produced. This also occurs with light, which is why objects under water appear to be shifted from their actual position. Refraction takes place at an interface due to the different velocities of the acoustic waves within the two materials, which are determined by the material properties for each material. Consider a wave traveling in one material and then entering a second material that has a higher acoustic velocity. When the wave front encounters the interface between these two materials, the portion of the wave that is already in the second material is moving faster than the portion of the wave in the first material. This causes the wave to bend.

Snell's Law describes the relationship between the angles and the velocities of the waves. When the angle of incidence is such that the ratio of longitudinal wave velocities indicates an angle of refraction of 90 degrees, surface waves are generated which follow the surface of a relatively thick solid material penetrating to a depth of one wavelength. These surface waves are often referred to as Rayleigh waves, and they combine both a longitudinal and transverse motion to create an elliptic particle motion with the major axis of the ellipse perpendicular to the surface of the solid. As the depth of an individual particle from the surface increases, the width of its elliptical motion decreases. Rayleigh waves are generated when a longitudinal wave intersects a surface near the second critical angle. Rayleigh waves are useful because they are very sensitive to surface defects (and other surface features) and they follow the surface around curves. Because of this, Rayleigh waves can be used to inspect areas that other waves might have difficulty reaching. Rayleigh waves propagating along solid surfaces in contact with a fluid such as a compliant gel boot will “leak” acoustic energy back into the fluid at the same critical angle. This damps the Rayleigh waves, but their propagation is otherwise as described above.

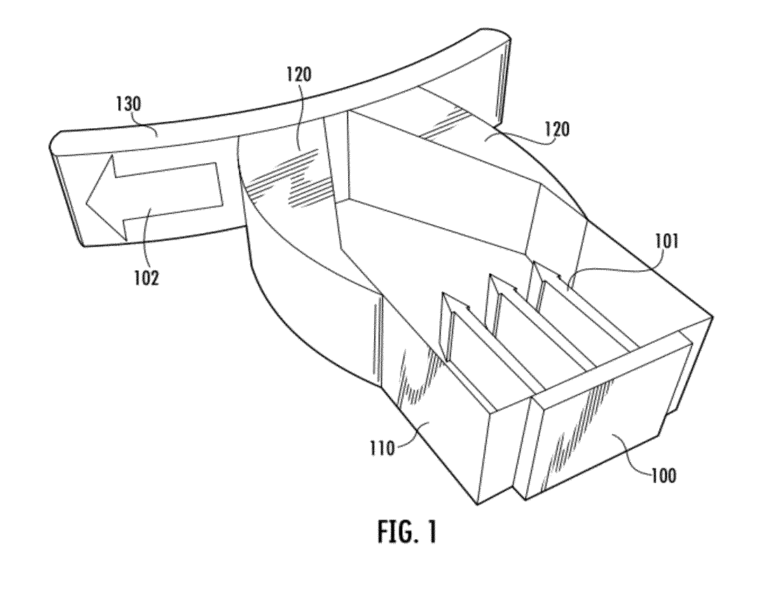

FIG. 1 depicts the inside of the distal portion of a dental handpiece which contains an ultrasound transducer 100 with a frequency of approximately 0.5 MHz to 100 MHz, preferably 10 MHz to 75 MHz. The frequency directly corresponds to the depth sensitivity of the surface acoustic waves, and thus frequency selection is critical to the functioning of the device. The ultrasound transducer 100 is attached to the back of a solid delay line 110 made from an appropriate material, including, but not limited to, for example, quartz and acrylic. The transducer sends ultrasound waves 101 towards the tooth, as depicted by the three shaded arrows in FIG. 1, which take distinct paths due to the shape of the delay line. The middle portion of the ultrasound beam is reflected by the flat middle portion of the end of the delay line, which is in contact with the tooth surface 130, and the resulting echo is recorded by the transducer to give information about the tooth surface at that particular location. The outer portions of the ultrasound beam are refracted across the angled sides of the tapered delay line which yields corresponding ultrasound waves 102 traveling through the aqueous material of the compliant gel boot 120 and proceeding circumferentially around the tooth. Wave 102 is shown traveling clockwise around the surface of the tooth. Another ultrasound wave not shown in FIG. 1 travels counterclockwise around the surface of the tooth. The compliant gel boot of the device is intended to optimize the coupling of ultrasound energy into and back out of the tooth surface, and is necessary because any air gaps at MHz frequencies will prevent ultrasound transmission.

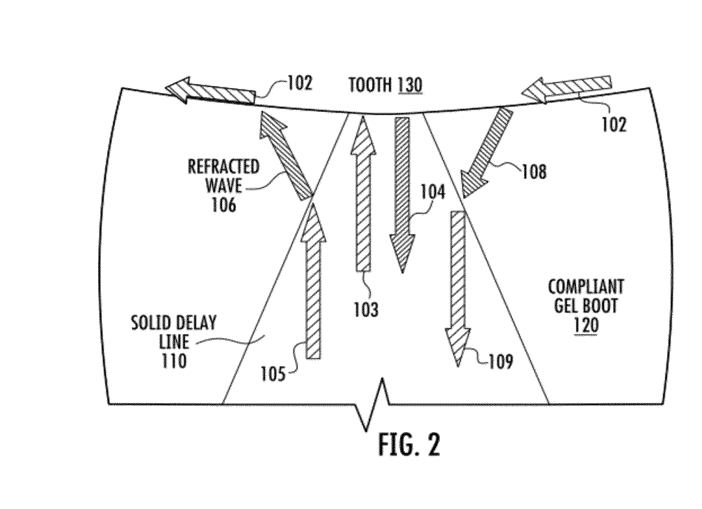

The angle of the taper on the delay line 110 is selected such that the refracted waves inside the compliant gel boot are then incident upon the tooth surface at or beyond the critical angle necessary for generation of surface acoustic waves in the enamel (and/or dentin) of the tooth. Because the plane of symmetry of the delay line is vertical, the surface acoustic waves will be circumferential in nature, traveling both clockwise and counter-clockwise. As the surface acoustic waves complete one or more circumferential paths, they will be recorded by the device because they will leak energy into the compliant gel boot at the same critical angle and thus will be refracted back into the solid tapered delay line directly towards the transducer. These various propagation paths are shown in FIG. 2. The transducer sends ultrasonic waves inside of the solid delay line 110 towards the tooth 130. Arrow 103 depicts the central part of the ultrasonic beam which reflects from the flat part of the delay line at the tooth surface and returns an echo 104 directly to the transducer. Arrow 105 corresponds to the part of the ultrasonic beam that refracts across the tapered side of the delay line into the surrounding compliant gel boot 120. The refracted wave 106 is at an angle to the tooth surface so as to convert into a surface wave 102 that travels around circumferentially following the surface contour of the tooth and is converted back to an acoustic wave 108 which gives another echo 109 which is recorded by the transducer.

An alternative embodiment of the invention uses a curved delay line rather than a straight taper. This approach refracts acoustic waves into the compliant gel boot at a range of angles so that the required critical angle for surface acoustic waves is less sensitive to a particular orientation of the handpiece.

Another embodiment utilizes a phased array of transducer sub-elements in order to focus and steer the beam. This can enable more accurate sizing and localization of flaws.

Another embodiment utilizes a broad-banded transducer which can excite waves of different frequencies since the depth sensitivity of the surface acoustic waves depends on frequency. Higher frequency waves are most sensitive to surface features; lower frequency waves are more sensitive to subsurface features. Measurements done sequentially at higher and then lower frequencies would allow flaws at the enamel surface to be better distinguished from flaws in the dentin or at the enamel-dentin interface.

Surface acoustic waves have the property that they follow the surface contour, so they can propagate around the irregular and highly variable tooth geometry. They also have the property that they are most sensitive to material discontinuities near the surface, with the depth of sensitivity greater at lower frequencies. Material variations and/or flaws such as cracks and carries will disturb the propagation of the surface acoustic waves and will thus disrupt the symmetry in the counter-propagating circumferential wave packets which will cause a signal change as they are summed by the transducer. Because the measurements are time-resolved and the speed of propagation is known, the circumferential location of the flaws is discernable by analysis of distortions in the recorded echoes.

Surface acoustic waves are also sensitive to pre-carious lesions that exist as demineralized regions just beneath the surface of the tooth. If such a lesion can be detected before it erupts, the lesion can be re-mineralized, thereby preventing the cavity. Current practice entails probing visually discolored areas mechanically, which can result in the probe breaking through to the sub-surface lesion, at which point the cavity must be drilled and filled.

By adjusting the frequency and thus the depth sensitivity of the surface acoustic waves, surface crazing can be distinguished from more serious structural cracking. Vertical cracks that propagate into the root are a serious clinical problem, and typically require invasive endodontic procedures to salvage the tooth. Circumferential surface acoustic waves are particularly well-suited to detecting vertical cracks.

Rayleigh waves provide a surface wave approach that can enhance the resolution of surface and sub-surface defects. These ultrasonic modes follow the surface curvature of the teeth, and can be launched by touching the transducer at any accessible region so that the ultrasound beam traverses areas that are not easily accessible, such as interproximal sites.

For each tooth, the probe tip is touched to the side of the tooth and one or more waveform signals are recorded and analyzed by the embedded computer software. In one embodiment, the ultrasound transducer is operated by a foot pedal. In B-mode imaging for soft tissues such as the heart or womb, a real-time display is presented to a health care professional to interpret. This traditional pulse-echo ultrasound imaging as practiced in typical medical imaging situations is not appropriate for ultrasonic imaging of teeth because the interproximal portions of the teeth are inaccessible. The interproximal portions of teeth are particularly problematic clinically because they are hidden from both direct view and mechanical probing.

Automated signal interpretation in lieu of scanning and imaging, respectively, makes the invention practical for routine dental practice. Advanced mathematics are utilized to identify reflections from the subtle lesions of interest in teeth. Additionally, simulations techniques enable better understand how ultrasonic waves propagate and interact with the inner complex structure of teeth.

Through the optimization of the clinically practical handpiece, see Example 1, the need arose for narrowly tailored artificial intelligence algorithms to automatically identify the very subtle echo-waveform features corresponding to the dental anatomy of interest. The wavelet transform is a mathematical operation which maps a waveform from time domain to time-scale domain, and the Dynamic Wavelet Fingerprinting (DWFP) technique is an implementation used herein and elsewhere to create binary contour plots of the wavelet transform coefficients. The DWFP technique allows for specific features to be identified in waveforms which correspond to specific identifiable interactions of the elastodynamic surface waves with specific flaws in teeth. Since small flaws are often of most interest, the waveform “echoes” are often subtle.

DWFP has previously shown promise for ultrasonographic periodontal probes as well as for a wide variety of other applications under current study. The resulting fingerprint-like image can be processed using similar techniques to human fingerprint analysis. Features are extracted from the shapes of the fingerprints and the feature vectors formed from those features are used for pattern classification to relate fingerprint features to tooth lesion characteristics.

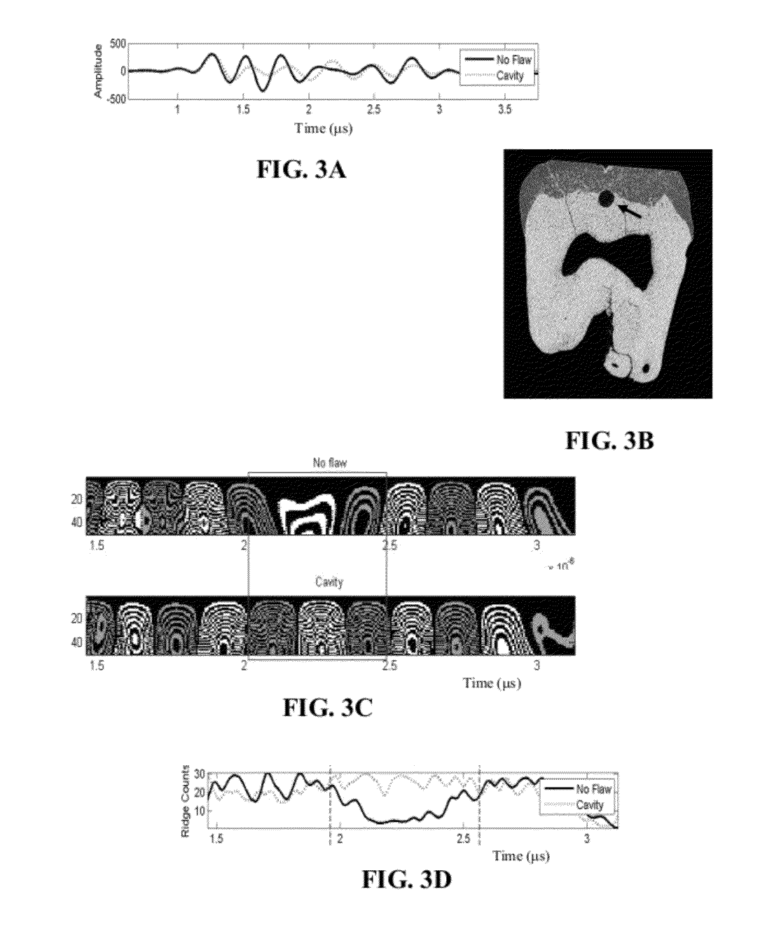

FIG. 3A shows in vitro data, for human teeth both with and without flaws, acquired with a contact transducer mounted in an ultrasound handpiece as described in Example 1. Although the two waveforms in FIG. 3A are clearly different, the differences are subtle and difficult to identify because of the presence of many echoes in the ultrasonic waveforms. FIG. 3B shows a slice of a CT-scan of a flawed tooth (with the flaw marked by the arrow). FIG. 3C shows DWFP fingerprint comparison of the flawed and unflawed locations on the tooth. The DWFP in FIG. 3C highlights differences between the flaw/no-flaw cases, and by having the computer count the number of fingerprint ridges as shown in FIG. 3D, a quantitative metric is established to automatically identify the presence of a tooth flaw. FIG. 3D is a graph showing the ridge counts as a function of depth for flawed and unflawed tooth sites, which provides a metric to discern the presence of flaws in the ultrasonography echoes. Data for FIGS. 3A-3D was collected from the experiment described in Example 1.

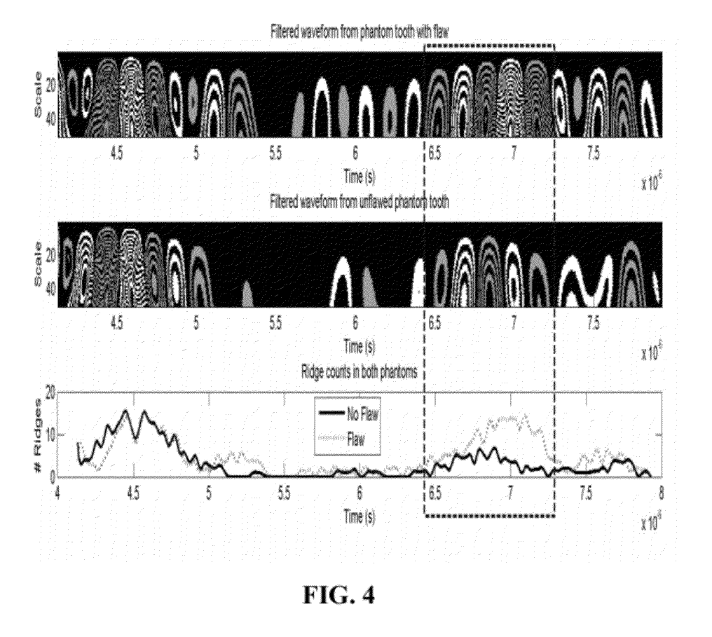

FIG. 4 shows DWFP fingerprints corresponding to a filtered waveform from a phantom tooth with flaw (top), a filtered waveform from an unflawed phantom tooth (middle), and, at bottom, a graph showing ridge counts as a function of time for the flawed and unflawed teeth. The abstract ridge-count metric in the DWFP allows the flaws to be identified automatically. Data for FIG. 4 was collected from the experiment described in Example 2.

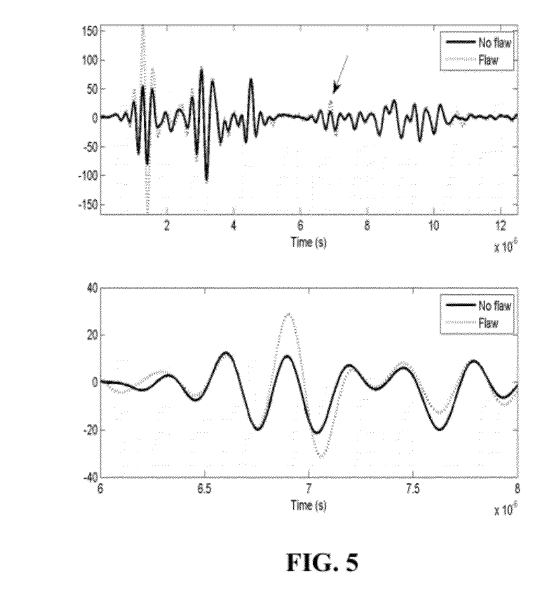

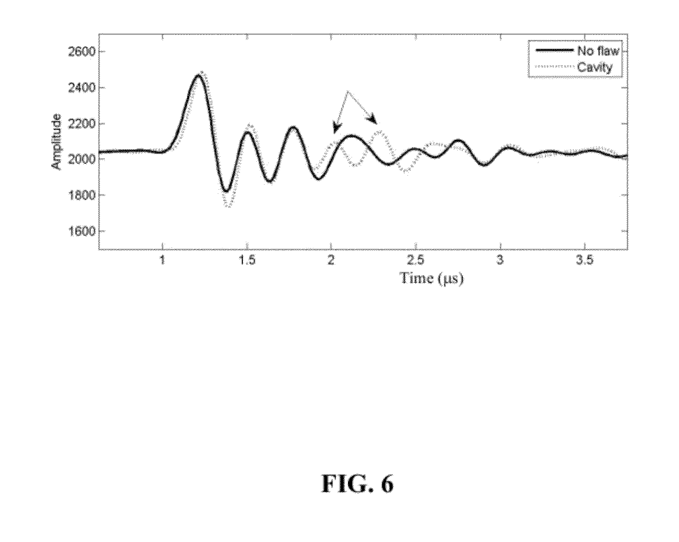

Comparison of the average waveforms over nine sites for both flawed and unflawed cases are shown in FIG. 5, which includes a zoomed-in version (bottom graph) that highlights the difference between the flawed and unflawed waveforms. Data for FIG. 5 was collected from the experiment described in Example 2. FIG. 6 is a graph showing the waveform comparison from flawed and unflawed sites of the same human tooth. Data for FIG. 6 was collected from the experiment described in Example 2.

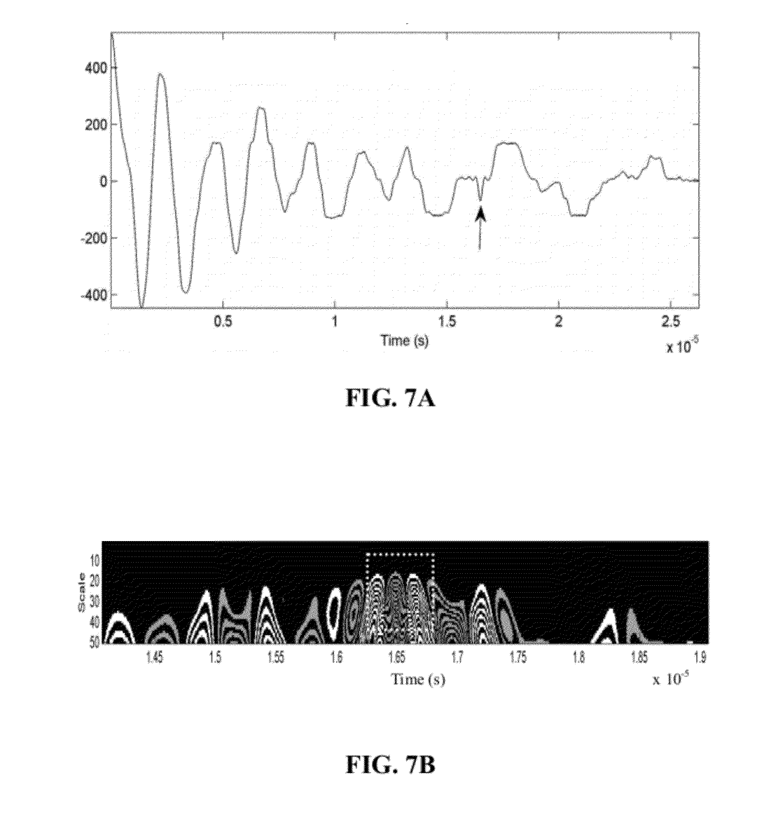

FIG. 7A shows the generation of surface waves modes in an aluminum plate, see Example 3. The wavelet fingerprint detects the small-amplitude aspect. Surface wave reflection from the defect is indicated with an arrow. The wavelet fingerprint region of interest in the box in FIG. 7B corresponds to the surface wave indicated by an arrow in the waveform shown in FIG. 7A. The associated wavelet fingerprint is centered on that surface wave reflection and highlights the unique characteristic features that can be detected with the DWFP's time-and-frequency analysis method. Data for FIG. 7 was collected from the experiment described in Example 3.

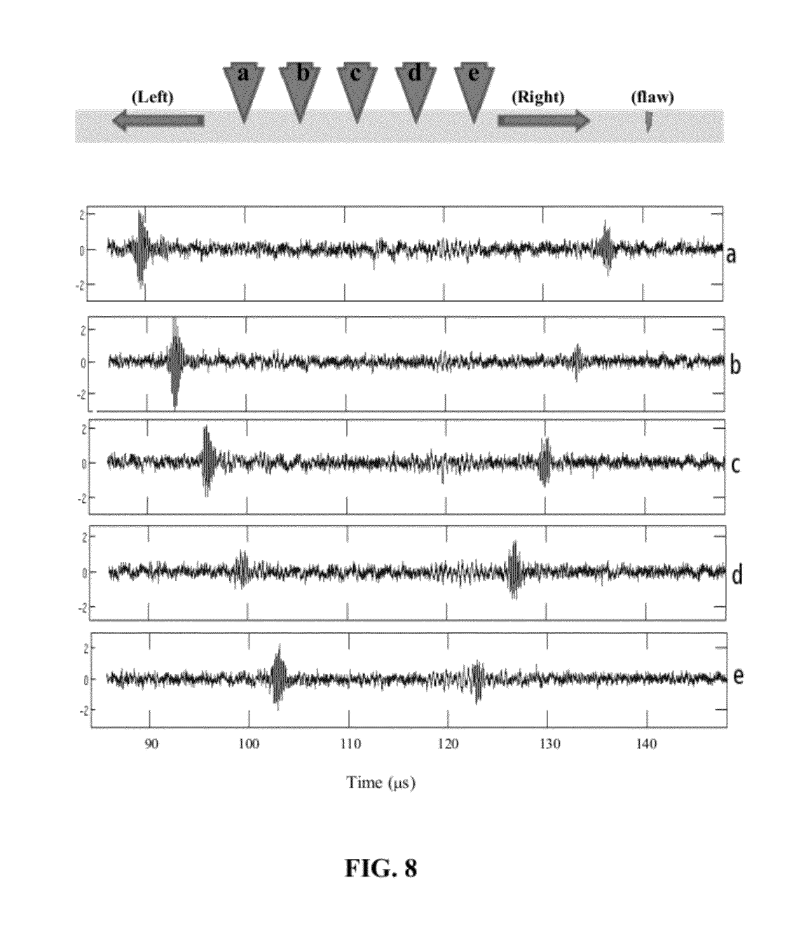

FIG. 8 shows five different measurement locations, with the corresponding Rayleigh wave reflections, for a flat sample with a slot flaw, see Example 3. The first pulse in each waveform is the left-traveling Rayleigh wave which has reflected from the left edge of the sample. It shows up later and later in time as the transducer steps farther away from the left edge because the propagation path-length increases. The second pulse is the flaw reflection due to the right-traveling Rayleigh wave which shifts earlier in time as the transducer is stepped further to the right because the transducer gets closer and closer to the flaw.

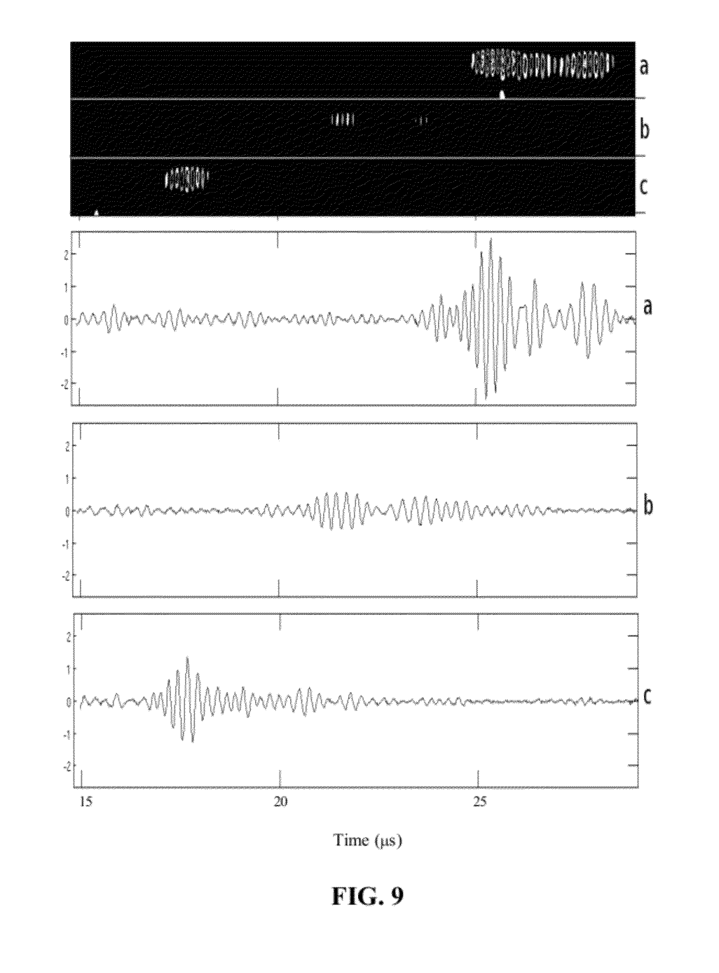

FIG. 9 shows three wavelet finger-prints with corresponding wave-forms showing Rayleigh wave reflections from cracks in a curved ceramic sample at three different distances from the transducer, see Example 3. Note that the wavelet fingerprint method both locates the flaws by keeping track of their respective time delays (“a” is farthest, “c” is nearest) but there is also a wealth of information in the fingerprint images about the strength and character of the reflection. Note the increased ridge count for the larger amplitude reflections. This sort of fingerprint minutiae, in addition to features such as shape, orientation, eccentricity, etc., is important in quantifying automatically the severity of different types of flaws. Pattern classification allows us to formally optimize the process.

After applying the wavelet fingerprint operation to the waveforms, the next step is to apply image recognition techniques to measure several properties of each fingerprint. In addition to ridge counting as depicted in FIG. 3D and FIG. 4, other techniques can be used, including fitting an ellipse matching the second moments of each fingerprint and subsequently measuring such aspects as eccentricity and orientation. Other properties measured include coefficients of second- or fourth-order polynomials fitting the boundary of the fingerprint, or simpler measurements like fingerprint area or height.

To select features of interest, we look for values of the fingerprint properties that are different for different types of flaws, yet consistent across each flaw classification. To this end, the mean and standard deviation of the feature vector for all tooth sites that correspond to each flaw is first plotted. Discrete points of the mean values of the feature vector are selected for the classifier whenever that value differs for different flaw measurements but where the standard deviation remains small. There is also the additional dimension in the re-sampled waveform for each tooth site. This creates three possible ways of separating the feature vectors into training and testing data sets. One approach is to average both over N (where N is in the range 30 to 50) repeated waveforms as well as averaging over all the tooth sites before selecting features of interest in the feature. Another approach is to avoid reducing dimensionality until after classification. In this case, the features are selected by averaging over one of the N waveforms at a time for all tooth sites with each of M flaws. This results in N different feature vectors for each tooth site. Classification is performed separately on each of those N feature vectors using standard classification maps, and dimensionality reduction can then occur after classification by combining the labels of each of the N repeated waveforms. The third way of selecting features involves using the training data set that was created in the first method, by averaging over the N waveforms, and the testing data set from the second method, by selecting feature vectors for each of the N waveforms individually. This again creates an array of possible labels for each tooth site, which are reduced after classification, usually by finding the mode of the labels.

Once the features have been generated for the wavelet fingerprint properties, standard pattern classification techniques are applied, e.g., from the PRTools catalog of MATLAB functions, many of which are Bayesian techniques which allow incorporation of a priori knowledge about flaws based on clinical history, etc. Combining classifiers can often reduce the error of pattern classifiers but, of course, can never substitute for proper classification techniques being applied when the individual classifiers are formed.

### EXAMPLES

The examples that follow are intended in no way to limit the scope of this invention but instead are provided to illustrate representative embodiments of the present invention. Many other embodiments of this invention will be apparent to one skilled in the art.

### Example 1

In order to demonstrate the concept, a standard Harrisonic NDT ultrasound angle block was modified. Tests were performed at four frequencies with varying amounts of the tip end removed in order to isolate the effects of interference between the surface waves and the directly reflected bulk waves. Coupling to both flat and curved surfaces was first accomplished by partial immersion and then by placing a finger cot (filled with Parker Aquasonic gel couplant) over the angle block for dry coupling. Parker Aquaflex ultrasound gel pads were also used for dry coupling of the ultrasound. Signals can be processed using refinements of our existing techniques used for ultrasonography of teeth, as described herein.

### Example 2

A preliminary study was conducted to demonstrate the ability to detect flaws in human and phantom teeth using a freehand ultrasonic handpiece intended to be compatible with clinical applications. A commercially-available Sonopen handpiece (available from Olympus NDT Inc., Waltham, Mass.) operating at 10 MHz was utilized to collect ultrasonographic data. The Sonopen handpiece uses a hard plastic tapered delay line to couple ultrasound into and back out of the tooth. In order to assist coupling to the hard tooth surfaces, the delay line was coated with a single layer of latex mold. The instrument itself is operated by foot pedal and is fully automated by computer.

Two phantom teeth of the same shape were selected for testing. Phantom teeth are model teeth, e.g., hard plastic teeth accurate in dimension and size. A flaw was introduced in one of the teeth, and the occlusal surface of both teeth were probed at nine different sites along the same direction using the handpiece with medical ultrasound gel for additional coupling. In these tests, a surface-skimming longitudinal wave was likely generated rather than a surface wave, as the orientation was perpendicular to circumferential, i.e., from the occlusal surface towards the tooth root. The waveforms over the tooth sites were averaged in order to eliminate the transient influences of noise and hand stability. The resulting comparison, presented in FIG. 5, shows a subtle but clear difference in the RF echo between the flawed and unflawed phantom teeth.

However, the reflections are subtle and features due to flaws are difficult to meaningfully distinguish by eye alone. Therefore, the Dynamic Wavelet Fingerprinting technique which generates 2D binary images from the RF waveforms becomes critical in order to enhance the possibility of feature detection. A useful feature extracted from the fingerprints is the number of ridges. FIG. 4 shows the ridge count twice smoothed and plotted below the fingerprints. The region of interest between about 6.25 seconds and about 7.25 seconds demonstrates a measured difference between the flawed and unflawed samples.

The same process can be performed for the human cadaver teeth. The waveform comparison of FIG. 6 was taken from flawed and unflawed sites of the same human tooth, with the arrows indicating features of interest. Note that the human cadaver RF echoes are less smooth than those from the phantom.

### Example 3

A preliminary study was conducted to demonstrate the ability to generate surface wave modes on the tooth's exterior in order to detect flaws on and near the surface. An aluminum plate with a small pit was utilized to generate the DWFP shown in FIG. 7. A flat sample with a slot flaw was utilized to generate the waveform shown in FIG. 8. A curved ceramic sample with cracks at three different distances from the transducer was utilized to generate the DWFP shown in FIG. 9.

### Incorporation by Reference

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

The articles “a” and “an” are used herein to refer to one or to more than one (i.e., to at least one) of the grammatical object of the article. By way of example, “a wave” means one wave or more than one wave.

Any ranges cited herein are inclusive.

1. A method for ultrasonic imaging of teeth comprising:
(A) passing a megahertz frequency through an interface of two materials with different indices of refraction;
(B) generating counter-propagating circumferential surface waves which follow the surface of the tooth in vivo; and
(C) identifying surface and subsurface defects in said tooth by analyzing the echo from said surface waves.

2. The method of claim 1, wherein said surface and subsurface defects disrupt the symmetry in said counter-propagating circumferential surface waves causing a variation in said echo.

3. The method of claim 1, wherein said surface waves can propagate to interproximal sites, following the curvature of the tooth.

4. The method of claim 1, wherein the first of said two materials contacts an accessible location on a locally flat portion of said tooth.

5. The method of claim 4, wherein the second of said two materials contacts a transducer.

6. The method of claim 5, wherein said transducer emits said megahertz frequency.

7. The method of claim 1, wherein said megahertz frequency is between 10 MHz and 75 MHz.

8. The method of claim 1, wherein said megahertz frequency is adjusted to provide greater depth sensitivity of said surface wave.

9. The method of claim 1, wherein the step of identifying surface and subsurface defects in said tooth by analyzing the echo from said waves comprises transforming said echo into a binary contour plot.

10. The method of claim 9, wherein said binary contour plot has an increased ridge count for the larger amplitude reflections.

11. The method of claim 9, wherein the features of said binary contour plot quantify the severity of different types of flaws.

12. The method of claim 1, wherein said surface and subsurface defects is selected from the group consisting of cracks, pre-carious lesions and carries.

13. The method of claim 1, wherein the step of identifying surface and subsurface defects in said tooth by analyzing the echo from said waves comprises analyzing the respective time delays in order to determine the circumferential location of said defect.
